SEQUENCE LISTING (27 sequences)

SEQ ID NO: 1 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 2 - protein, 774 aa, Artificial Sequence
  Exonuclease deficient mutant of Vent polymerase (Thermococcus litoralis)
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 3 - protein, 5 aa, Artificial Sequence
  Conserved Exonuclease domain sequence of DNA polymerases
FDIET

SEQ ID NO: 4 - protein, 5 aa, Artificial Sequence
  Mutated FDIET conserved exonuclease motif.
FAIAT

SEQ ID NO: 5 - DNA, 41 bp, Artificial sequence
  Oligonucleotide primer for genetic profiling
cgacgtatgcgtaacccgtatcgttttttttttttttttvn

SEQ ID NO: 6 - DNA, 27 bp, Artificial Sequence
  Oligonucleotide primer for genetic profiling
gcggcgcctatcttactatccagacta

SEQ ID NO: 7 - DNA, 24 bp, Artificial Sequence
  Oligonucleotide primer for genetic profiling
cgctcgtagtcgaacgcctaacca

SEQ ID NO: 8 - DNA, 20 bp, Artificial Sequence
  Oligonucleotide primer for genetic profiling
ccatacgacgtcccagacta

SEQ ID NO: 9 - DNA, 24 bp, Artificial Sequence
  Oligonucleotide primer for genetic profiling
agctctagcatttaggtgacacta

SEQ ID NO: 10 - DNA, 24 bp, Artificial Sequence
  Oligonucleotide primer for genetic profiling
aagcctcccattagttttccatta

SEQ ID NO: 11 - DNA, 24 bp, Artificial Sequence
  Oligonucleotide primer for genetic profiling
cacaacagcatcaccatagtcacc

SEQ ID NO: 12 - DNA, 24 bp, Artificial Sequence
  Oligonucleotide primer for genetic profiling
acgtcagctgcagcctattttgtt

SEQ ID NO: 13 - DNA, 24 bp, Artificial Sequence
  Oligonucleotide primer for genetic profiling
ttgtcctggcgctatttgtcttac

SEQ ID NO: 14 - DNA, 20 bp, Artificial Sequence
  Oligonucleotide primer for genetic profiling
aagcagaggcggcagtcaag

SEQ ID NO: 15 - DNA, 20 bp, Artificial Sequence
  Oligonucleotide primer for genetic profiling
tagcgcgagggcagtttcac

SEQ ID NO: 16 - DNA, 20 bp, Artificial Sequence
  Oligonucleotide primer for genetic profiling
ctcgtacccgctcaatgtgg

SEQ ID NO: 17 - DNA, 19 bp, Artificial Sequence
  Oligonucleotide primer for genetic profiling
acgctgcgacgctcctaat

SEQ ID NO: 18 - DNA, 22 bp, Artificial Sequence
  Oligonucleotide primer for genetic profiling
tgccatgcaaaaggagagtgat

SEQ ID NO: 19 - DNA, 24 bp, Artificial Sequence
  Oligonucleotode primer for genetic profiling
ctgacaacaatggtgcaagtaaga

SEQ ID NO: 20 - DNA, 20 bp, Artificial Sequence
  Oligonucleotide primer for genetic profiling
gttacctactgagtaggcgg

SEQ ID NO: 21 - DNA, 26 bp, Artificial Sequence
  Oligonucleotide primer for genetic profiling
tgaaggatacacaccacatatcccat

SEQ ID NO: 22 - DNA, 18 bp, Artificial Sequence
  Oligonucleotide primer for genetic profiling
cacccacactgtgcccat

SEQ ID NO: 23 - DNA, 18 bp, Artificial Sequence
  Oligonucleotide primer for genetic profiling
tggtggtgaagctgtagc

SEQ ID NO: 24 - DNA, 22 bp, Artificial Sequence
  Oligonucleotide primer for genetic profiling
gccccagctcaatcaagtccta

SEQ ID NO: 25 - DNA, 19 bp, Artificial Sequence
  Oligonucleotide primer for genetic profiling
caccctgcagcccaagttc

SEQ ID NO: 26 - DNA, 22 bp, Artificial Sequence
  Oligonucleotide primer for genetic profiling
cccccagatgacagcaccagaa

SEQ ID NO: 27 - DNA, 21 bp, Artificial Sequence
  Oligonucleotide primer for genetic profiling
cacacggggctcgacaggaag

(19) United States
(12) Patent Application Publication
(10) Pub. No.: US 2006/0269932 A1
(43) Pub. Date: Nov. 30, 2006

(54) Compositions and methods for polynucleotide amplification and detection

(75) Inventors: Vladimir I. Slepnev, Newton, MA (US); Elizabeth Garcia, Barrington, RI (US)

Correspondence Address: EDWARDS ANGELL PALMER & DODGE LLP, P.O. Box 55874, Boston, MA, 02205, US

(73) Assignee: Primera BioSystems, Inc.

(21) Appl. No.: 11/300,795

(22) Filed: Dec. 15, 2005

Related U.S. Application Data
(60) Provisional application No. 60/636,770, filed on Dec. 15, 2004.

(51) Int. Cl. C12Q 1/68; C12P 19/34

(52) U.S. Cl. 435/6; 435/91.2

(57) ABSTRACT

Described are characteristics rendering DNA polymerases well suited for single target and multiplex genetic profiling methods, and polymerases having these characteristics. Also described are methods of single target and multiplex analyses using polymerases having the described characteristics.

The present application claims priority under 35 U.S.C. 119(e) to U.S. Provisional Application Ser. No. 60/636,770, filed Dec. 15, 2004, the entirety of which is hereby incorporated by reference.

### BACKGROUND OF THE INVENTION

The amplification of nucleic acids through the use of enzymes, particularly thermostable enzymes, has revolutionized the analysis of nucleic acid samples. Generally, the process involves the hybridization of one or more nucleic acid primers to a nucleic acid template, followed by the template-dependent extension of the primer(s) by one or more enzymes in the presence of nucleoside triphosphates or their functional equivalent. The polymerase chain reaction (PCR), for example, finds use in nucleic acid cloning for basic research, a wide array of clinical, agricultural and environmental diagnostic analyses, and in areas such as forensics.

One area that was largely made possible by nucleic acid amplification technology is genetic profiling, the identification and comparison of patterns of genetic sequences and their expression between individuals, populations or groups of individuals and between species. Genetic profiling is useful in the context of genomic comparisons, for example comparisons of the genomic makeup of an individual relative to a population, and in the context of expression profiling, in which the expression pattern of one or more sequences is analyzed relative to a standard or to a population. Variations in the sequence and/or expression of nucleic acid sequences are presently being exploited in an effort to enhance the prediction and diagnosis of disease. Similarly, the field of pharmacogenomics seeks to identify variations in genetic sequence that correlate with differing responses to therapeutic approaches, particularly drug responses.

Most genetic profiling methods rely upon nucleic acid amplification for at least one part of the process, and most of these rely upon thermostable enzymes. High fidelity of the polymerase enzyme(s) used is critical to the process, particularly where sequences frequently differ by as little as one nucleotide base in a given stretch of sequence (a single nucleotide polymorphism, or SNP). The efficiency of polymerization is also important. Polymerase enzyme efficiency relates to the processivity and thermal stability of the enzyme, among other factors.

One of the major attributes of the PCR process is its speed, often amplifying target sequences within minutes to hours. However, it can be useful to monitor the progress of the reaction. This is especially true for quantitative PCR methods, which seek to correlate the abundance of a detectable PCR product with the abundance of the template in the sample from which it was amplified. In those methods, because PCR amplification reaches a plateau or stationary phase in which the abundance of product no longer reflects the abundance of original template, and because sequence-specific variations in amplification efficiency are magnified by the process itself, it can be important to be able to visualize the amount of a target product at a given point in the amplification.

The monitoring of an amplification reaction's progress permits more accurate quantification of starting target DNA concentrations in multiple-target amplifications, because the relative values of close concentrations can be resolved by taking into account the history of the relative concentration values during the reaction. Such real time monitoring also permits the efficiency of the amplification reaction to be evaluated, which can indicate whether reaction inhibitors are present in a sample.

Capillary electrophoresis has been used to quantitatively detect gene expression. Rajevic at el. (2001, Pflugers Arch. 442(6 Suppl 1):R190-2) discloses a method for detecting differential expression of oncogenes by using seven pairs of primers for detecting the differences in expression of a number of oncogenes simultaneously. Sense primers were 5′ end-labelled with a fluorescent dye and multiplex fluorescent RT-PCR results were analyzed by capillary electrophoresis on an ABI-PRISM 310 Genetic Analyzer. Borson et al. (1998, Biotechniques 25:130-7) describes a strategy for dependable quantitation of low-abundance mRNA transcripts based on quantitative competitive reverse transcription PCR (QC-RT-PCR) coupled to capillary electrophoresis (CE) for rapid separation and detection of products. George et al., (1997, J. Chromatogr. B. Biomed. Sci. Appl. 695:93-102) describes the application of a capillary electrophoresis system (ABI 310) to the identification of fluorescent differential display generated EST patterns. Odin et al. (1999, J. Chromatogr. B. Biomed. Sci. Appl. 734:47-53) describes an automated capillary gel electrophoresis with multicolor detection for separation and quantification of PCR-amplified cDNA.

There are three major families of DNA polymerases, termed families A, B and C. The classification of a polymerase into one of these three families is based on structural similarity of a given polymerase to E. coli DNA polymerase I (Family A), II (Family B) or III (Family C). As examples, Family A DNA polymerases include, but are not limited to Klenow DNA polymerase, Thermus aquaticus DNA polymerase I (Taq polymerase) and bacteriophage T7 DNA polymerase; Family B DNA polymerases, formerly known as α-family polymerases (Braithwaite and Ito, 1991, Nuc. Acids Res. 19:4045), include, but are not limited to human α, δ and ε DNA polymerases, T4, RB69 and φ29 bacteriophage DNA polymerases, and Pyrococcus furiosus DNA polymerase (Pfu polymerase); and Family C DNA polymerases include, but are not limited to Bacillus subtilis DNA polymerase III, and E. coli DNA polymerase III α and ε subunits (listed as products of the dnaE and dnaQ genes, respectively, by Brathwaite and Ito, 1993, Nucleic Acids Res. 21: 787). An alignment of DNA polymerase protein sequences of each family across a broad spectrum of archaeal, bacterial, viral and eukaryotic organisms is presented in Braithwaite and Ito (1993, supra), which is incorporated herein by reference. Hopfner et al. also aligned the sequences of polymerases encoded by Thermococcus gorgonarius, Pyrococcus furiosus, Thermococcus litoralis, Methanococcus voltae, bacteriophage RB69, bacteriophage T4, and E. coli pol II and human pol δ.

### SUMMARY OF THE INVENTION

The invention relates to the identification of nucleic acid polymerases that have appropriate characteristics for genetic profiling methods, and relates to genetic profiling methods that use those polymerases. In particular, the invention relates to the identification of nucleic acid polymerases suitable for profiling methods that rely upon thermostable polymerases. The invention also relates to the identification of thermostable polymerases that are well suited for genetic profiling methods involving the use of 5′ labeled primers, amplification of multiple templates in a single reaction (so called “multiplex reactions”), or both.

In one aspect, a method is provided for the analysis of a nucleic acid, the method involving: I) providing a reaction mixture by contacting a nucleic acid template with an oligonucleotide primer and a thermostable DNA polymerase that is at least as processive as VentExo⁻™ DNA polymerase, under conditions that permit the hybridization of the primer to the template. Under these conditions, the nucleic acid template and the oligonucleotide primer are hybridized. Following the hybridization, at least two cycles as follows are performed: a) template-dependent extension of the primer by the DNA polymerase; b) sampling the reaction mixture; c) thermally separating the nucleic acid strands in the reaction mixture; and d) permitting the hybridization of the primer to a nucleic acid template. Nucleic acid molecules in the samples withdrawn during the cycling process are separated, e.g., by capillary electrophoresis, and the separated nucleic acids are detected, preferably both for size and for abundance. This process generates a curve representing the amplification profile for each amplified nucleic acid molecule or target. The respective curves or amplification profiles permit the sensitive determination, e.g., of the amount of template in the initial sample for a given amplicon.

In another aspect, a method is provided for the analysis of a nucleic acid, the method involving I) making a reaction mixture by contacting a nucleic acid template with an oligonucleotide primer and a 5′-3′ exonuclease-deficient, thermostable DNA polymerase with strand displacement activity, under conditions that permit the hybridization of the primer to the template. Under these conditions, the nucleic acid template and the oligonucleotide primer are hybridized. Following the hybridization, at least two cycles as follows are performed: a) template-dependent extension of the primer by the DNA polymerase; b) sampling the reaction mixture; c) thermally separating the nucleic acid strands in the reaction mixture; and d) permitting the hybridization of the primer to a nucleic acid template. Nucleic acid molecules in the samples withdrawn during the cycling process are separated, e.g., by capillary electrophoresis, and the separated nucleic acids are detected, preferably both for size and for abundance. This process generates a curve representing the amplification profile for each amplified nucleic acid molecule or target. The respective curves or amplification profiles permit the sensitive determination, e.g., of the amount of template in the initial sample for a given amplicon.

In another aspect, a method is provided for the analysis of a nucleic acid, the method involving I) making a reaction mixture by contacting a nucleic acid template with an oligonucleotide primer and a 3′-5′ exonuclease-deficient, thermostable DNA polymerase with strand displacement activity, under conditions that permit the hybridization of the primer to the template. Under these conditions, the nucleic acid template and the oligonucleotide primer are hybridized. Following the hybridization, at least two cycles as follows are performed: a) template-dependent extension of the primer by the DNA polymerase; b) sampling the reaction mixture; c) thermally separating the nucleic acid strands in the reaction mixture; and d) permitting the hybridization of the primer to a nucleic acid template. Nucleic acid molecules in the samples withdrawn during the cycling process are separated, e.g., by capillary electrophoresis, and the separated nucleic acids are detected, preferably both for size and for abundance. This process generates a curve representing the amplification profile for each amplified nucleic acid molecule or target. The respective curves or amplification profiles permit the sensitive determination, e.g., of the amount of template in the initial sample for a given amplicon.

In one embodiment of either aspect, the reaction mixture is a PCR reaction mixture. For each template sequence, whether known or unknown, a PCR reaction mixture comprises a nucleic acid primer that hybridizes upstream of the sequence to be amplified and a nucleic acid primer that hybridizes downstream of the sequence to be amplified and on the opposite strand from the upstream primer, such that the extension of each hybridized primer generates another template for the hybridization of the other primer in subsequent rounds of extension.

In one embodiment of either aspect, the reaction mixture contains a set of at least 5 oligonucleotide primers, such that at least 5 different sequences are replicated in multiplex upon primer extension. In a preferred embodiment the reaction comprises a pair of PCR primers for each of 5 or more different sequences to be analyzed. In another preferred embodiment, there are 10 or more pairs of PCR primers, and the reaction mixture permits multiplex amplification of at least 10 different sequences. In other embodiments, the reaction mixture comprises primer sets that permit the multiplex amplification of 15, 20, 25, 30, 35, 40, 50, 75, 100 or more templates in a single reaction.

In another embodiment, the polymerase is one of the following enzymes: Vent Exo⁻™ DNA polymerase (Vent Exo-™ is clearly a trade name for an exonucleases deficient mutant of wild-type Vent™ polymerase (described at GenBank Accession No. P30317)—for the avoidance of doubt, Vent Exo-™ polymerase has the sequence of SEQ ID NO: 2); Tfl DNA polymerase, 9° Nₘ DNA polymerase, and Tfu DNA polymerase.

In another embodiment, the reaction mixture of step (I) is a PCR reaction mixture that comprises primers specific for at least 5 or, alternatively, at least 10 different template nucleic acid sequences, wherein the 5′-3′ exonuclease-deficient, thermostable DNA polymerase has 3′ to 5′ exonuclease activity—that is, the enzyme is not 3′-5′ exonucleases deficient as the term is defined herein.

As used herein, the term “strand displacement activity” refers to the dissociation of a paired nucleic acid strand from its complementary strand by the advance of a polymerizing enzyme along the complementary strand as template. As used herein, the term “at least some strand displacement activity” means that, at the optimum temperature for a given DNA polymerase, that polymerase has at least 10%, and preferably at least 20%, 30%, 40%, 50%, 60%, 70%, 80%, 90%, 100% or more, e.g., 2×, 3×, etc. of the strand displacement activity of Thermococcus litoralis DNA polymerase (Vent™ polymerase; polypeptide sequence available at GenBank Accession No. AAA72101, SEQ ID NO: 1) measured at 72° C. according to the method described by Kong et al., 1993, J. Biol. Chem. 268: 1965-1975. It is preferred that the strand displacement activity, measured at optimal polymerizing temperature for the DNA polymerase, is at least as great as the strand displacement activity of Thermococcus litoralis DNA polymerase (Vent™ polymerase; SEQ ID NO: 1) measured at 72° C.

As used herein, the “efficiency” of a thermostable polymerase enzyme refers to the amount of product produced from a given amount of template in a given number of cycles. Thus, a thermostable polymerase that generates more copies of a template in, for example, 20 thermal cycles, than another thermostable polymerase generates from the same amount of template in 20 thermal cycles, is considered to be more “efficient” than the other. By “more efficient” is meant at least 5%, and preferably at least 10%, 20%, 50%, 75%, or even beyond 100% more efficient. The “efficiency” of a thermostable polymerase is a factor of, among other things, the processivity of the enzyme, and the thermal stability of the enzyme (that is, for how many thermal cycles does the enzyme retain activity?).

As used herein, the term “processive” refers to a measure of the number of nucleotides incorporated per initiation event by a nucleic acid polymerase. Where necessary, processivity is measured as described by Kong et al., 1993, J. Biol. Chem. 268: 1965-1975. As an example, the processivity of Vent polymerase measured in this assay is 7 nucleotides per initiation event.

As used herein, “T. litoralis DNA polymerase” encompasses a DNA polymerase sequence cloned from T. litoralis or a sequence derived by mutation of such a clone.

“Vent Exo⁻™” refers to the Exo⁻ Vent polymerase described in U.S. Pat. No. 5,322,785 and provided herein as SEQ ID NO: 2. Vent Exo⁻™ polymerase is expressed by E. coli NEB 681 as described, for example, in U.S. Pat. No. 5,322,785, and is available, e.g., from New England Biolabs, Ipswich, Mass. Vent™ DNA polymerase is the polymerase of SEQ ID NO: 1.

As used herein, the phrase “exonuclease-deficient” refers to a DNA polymerase that is deficient in both 5′-3′ and 3′-5′ exonuclease activities.

The term “deficient” when applied herein to 5′-3′ exonuclease activity (e.g., “5′-3′ exonuclease deficient”) means that the polymerase has less than 5%, per unit of polymerase activity, of the 5′ to 3′ exonuclease activity per unit of Taq polymerase activity.

The term “deficient” when applied herein to 3′ to 5′ exonuclease activity (e.g., “3′-5′ exonuclease deficient”) means that the polymerase has less than 5% of the 3′-5′ exonuclease activity, per unit of polymerase activity, of the 3′ to 5′ exonuclease activity of the T. litoralis DNA polymerase of SEQ ID NO: 1 (Vent DNA polymerase).

As used herein, the phrase “conditions that permit the hybridization” of a primer to a template refer to the temperature and buffer conditions (salt concentration(s), buffering agent(s), pH, divalent cation(s), etc.) present during the annealing step of a primer extension or PCR reaction using the given polymerase enzyme and primer or primers. Buffer conditions optimal for a given polymerase are nearly always recommended by the manufacturer; however, one of skill in the art can readily determine the buffer and temperature conditions for the annealing step in a primer extension or PCR reaction for a given template and enzyme.

As used herein, the term “primer extension” refers to the template-dependent extension of an oligonucleotide primer, annealed to a template nucleic acid molecule, by one or more nucleotides, the reaction catalyzed by a nucleic acid polymerase enzyme.

As used herein, the term “sampling” refers to the removal of an aliquot of an amplification reaction mixture from a PCR reaction during the thermal cycling regimen. A sample is less than the whole reaction volume, but can vary in volume depending, for example, upon how many samples are to be taken (e.g., varying cycle numbers can indicate greater numbers of samples to withdraw, which, unless initial reaction volume is increased, would dictate smaller samples) and how much volume is desired to be loaded onto the capillary electrophoresis capillary.

As used herein, the term “detecting nucleic acid molecules” refers to the detection of a value including, for example, the size, presence, absence and/or amount of a nucleic acid molecule.

As used herein, the term “plotting a value” refers to plotting of a co-ordinate physically, electronically or virtually.

As used herein, a “PCR reaction mixture” comprises, in addition to template nucleic acid, forward and back primers that anneal to opposite strands of a target sequence and a DNA polymerase as described herein.

As used herein, the term “capillary electrophoresis” means the electrophoretic separation of nucleic acid molecules in an aliquot from an amplification reaction wherein the separation is performed in a capillary tube. Capillary tubes are available with inner diameters from about 10 to 300 μm, and can range from about 0.2 cm to about 3 m in length, but are preferably in the range of 0.5 cm to 20 cm, more preferably in the range of 0.5 cm to 10 cm. In addition, the use of microfluidic microcapillaries (available, e.g., from Caliper or Agilent Technologies) is specifically contemplated within the meaning of “capillary electrophoresis.”

As used herein, the term “reverse transcription reaction” refers to an in vitro enzymatic reaction in which the template-dependent polymerization of a DNA strand complementary to an RNA template occurs. Reverse transcription is performed by the extension of an oligonucleotide primer annealed to the RNA template, and most often uses a viral reverse-transcriptase enzyme, such as AMV (avian myeloblastosis virus) reverse transcriptase, or MMLV (Moloney murine leukemia virus) reverse transcriptase or thermostable DNA polymerase capable of using RNA as a template (e.g. Tfh DNA polymerase). Conditions and methods for reverse transcription are well known in the art. Exemplary conditions for reverse transcription include the following: for AMV reverse transcriptase—reaction at 37-55° C. in buffer containing 50 mM Tris-HCl, pH 8.3, 75 mM KCl, 3 mM MgCl₂, 10 mM DTT, 0.8 mM dNTPs, 50 units of reverse transcriptase, and 1-5 Hg of template RNA and other additives (e.g BSA, glycerol, carbohydrates (glucose, trehalose); for MMLV reverse transcriptase—reaction at 37-55° C. in buffer containing 50 mM Tris-HCl, pH 8.3, 30 mM KCl, 8 mM MgCl₂, 10 mM DTT, 0.8 mM dNTPs, 50 units of reverse transcriptase, and 1-5 μg of template RNA and other additives (e.g BSA, glycerol, carbohydrates (glucose, trehalose)).

As used herein, the term “abundance of nucleic acid” refers to the amount of a particular target nucleic acid sequence present in a sample or aliquot. The amount is generally measured as a relative amount in terms of concentration or copy number of the target sequence relative to the amount of a standard of known concentration or copy number. Alternatively, the amount in one unknown sample is measured relative to the amount in another unknown sample. As used herein, abundance of a nucleic acid is measured on the basis of the intensity of a detectable label, most often a fluorescent label. The methods of the invention permit one to extrapolate the relative amount of one or more target sequences in a nucleic acid sample from the amplification profile of that target sequence or sequences from that sample.

As used herein, the term “amplified product” refers to polynucleotides which are copies of a portion of a particular polynucleotide sequence and/or its complementary sequence, which correspond in nucleotide sequence to the template polynucleotide sequence and its complementary sequence. An “amplified product,” according to the invention, may be DNA or RNA, and it may be double-stranded or single-stranded.

As used herein, the term “distinctly sized amplification product” means an amplification product that is resolvable from amplification products of different sizes. “Different sizes” refers to nucleic acid molecules that differ by at least one nucleotide in length. Generally, distinctly sized amplification products useful according to the methods described herein differ by greater than or equal to more nucleotides than the limit of resolution for the separation process used in a given method according to the invention. For example, when the limit of resolution of separation is one base, distinctly sized amplification products differ by at least one base in length, but can differ by 2 bases, 5 bases, 10 bases, 20 bases, 50 bases, 100 bases or more. When the limit of resolution is, for example, 10 bases, distinctly sized amplification products will differ by at least 10 bases, but can differ by 11 bases, 15 bases, 20 bases, 30 bases, 50 bases, 100 bases or more.

As used herein, the term “profile” or the equivalent terms “amplification curve” and “amplification plot” mean a mathematical curve representing the signal from a detectable label incorporated into a nucleic acid sequence of interest at two or more steps in an amplification regimen, plotted as a function of the cycle number from which the samples were withdrawn. The profile is preferably generated by plotting the fluorescence of each band detected after capillary electrophoresis separation of nucleic acids in the individual reaction samples. Most commercially available fluorescence detectors are interfaced with software permitting the generation of curves based on the signal detected.

As used herein, the term “first strand cDNAs” means the products of a reverse transcription reaction.

As used herein, the term “distinguishably labeled” means that the signal from one labeled oligonucleotide primer or a nucleic acid molecule into which it is incorporated can be distinguished from the signal from another such labeled primer or nucleic acid molecule. Detectable labels can comprise, for example, a light-absorbing dye, a fluorescent dye, or a radioactive label. Fluorescent dyes are preferred. Generally, a fluorescent signal is distinguishable from another fluorescent signal if the peak emission wavelengths are separated by at least 20 nm. Greater peak separation is preferred, especially where the emission peaks of fluorophores in a given reaction are wide, as opposed to narrow or more abrupt peaks.

As used herein, the term “sample” refers to a biological material which is isolated from its natural environment and containing a polynucleotide. A “sample” according to the invention may consist of purified or isolated polynucleotide, or it may comprise a biological sample such as a tissue sample, a biological fluid sample, or a cell sample comprising a polynucleotide. A biological fluid includes blood, plasma, sputum, urine, cerebrospinal fluid, lavages, and leukophoresis samples. A sample of the present invention may be any plant, animal, bacterial or viral material containing a polynucleotide.

As used herein, an “oligonucleotide primer” refers to a polynucleotide molecule (i.e., DNA or RNA) capable of annealing to a polynucleotide template and providing a 3′ end to produce an extension product which is complementary to the polynucleotide template. The conditions for initiation and extension usually include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer according to the invention may be single- or double-stranded. The primer is single-stranded for maximum efficiency in amplification, and the primer and its complement form a double-stranded polynucleotide. “Primers” useful in the present invention are less than or equal to 100 nucleotides in length, e.g., less than or equal to 90, or 80, or 70, or 60, or 50, or 40, or 30, or 20, or 15, or equal to 10 nucleotides in length.

As used herein, the term “specifically hybridizes” means that under given hybridization conditions a probe or primer hybridizes only to the target sequence in a sample comprising the target sequence. Given hybridization conditions include the conditions for the annealing step in an amplification regimen, i.e., annealing temperature selected on the basis of predicted Tm, and salt conditions suitable for the polymerase enzyme of choice.

As used herein, the term “nucleic acid sequence of interest” or “target sequence” refers to a nucleic acid sequence, in a sample, for which one wishes to determine the presence or abundance. The sequence of interest will most often be a transcript of an RNA expression unit or gene, but can be any nucleic acid sequence, e.g., a sequence comprised in a viral, bacterial, fungal or higher eukaryotic genome, or an artificial or synthetic sequence.

As used herein, the term “amplification regimen” means a process of specifically amplifying the abundance of a nucleic acid sequence of interest. An amplification regimen according to the invention comprises at least two, and preferably at least 5, 10, 15, 20, 25, 30, 35 or more iterative cycles of thermal denaturation, oligonucleotide primer annealing to template molecules, and nucleic acid polymerase extension of the annealed primers. Conditions and times necessary for each of these steps are well known in the art. Amplification achieved using an amplification regimen is preferably exponential, but can alternatively be linear. An amplification regimen according to the invention is preferably performed in a thermal cycler, many of which are commercially available.

As used herein, the term “strand separation” means treatment of a nucleic acid sample such that complementary double-stranded molecules are separated into two single strands available for annealing to an oligonucleotide primer. Strand separation according to the invention is achieved by heating the nucleic acid sample above its Tₘ. Generally, for a sample containing nucleic acid molecules in buffer suitable for a nucleic acid polymerase, heating to 94° C. is sufficient to achieve strand separation according to the invention. An exemplary buffer contains 50 mM KCl, 10 mM Tric-HCl (pH 8.8@ 25° C.), 0.5 to 3 mM MgCl₂, and 0.1% BSA.

As used herein, the term “primer annealing” means permitting oligonucleotide primers to hybridize to template nucleic acid strands. Conditions for primer annealing vary with the length and sequence of the primer and are based upon the calculated Tₘ for the primer. Generally, an annealing step in an amplification regimen involves reducing the temperature following the strand separation step to a temperature based on the calculated Tₘ for the primer sequence, for a time sufficient to permit such annealing. Tₘ can be readily predicted by one of skill in the art using any of a number of widely available algorithms (e.g., Oligo™, Primer Design and programs available on the internet, including Primer3 and Oligo Calculator). For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can temperatures more than 5° C. below or above the predicted Tₘ (e.g., 6° C. below, 8° C. below, 10° C. below or lower and 6° C. above, 8° C. above, or 10° C. above). Generally, the closer the annealing temperature is to the Tₘ, the more specific is the annealing. Time of primer annealing depends largely upon the volume of the reaction, with larger volumes requiring longer times, but also depends upon primer and template concentrations, with higher relative concentrations of primer to template requiring less time than lower. Depending upon volume and relative primer/template concentration, primer annealing steps in an amplification regimen can be on the order of 1 second to 5 minutes, but will generally be between 10 seconds and 2 minutes, preferably on the order of 30 seconds to 2 minutes.

As used herein, the term “polymerase extension” means the template-dependent incorporation of at least one complementary nucleotide, by a nucleic acid polymerase, onto the 3′ end of an annealed primer. Polymerase extension preferably adds more than one nucleotide, preferably up to and including nucleotides corresponding to the full length of the template. Conditions for polymerase extension vary with the identity of the polymerase. The temperature of polymerase extension is based upon the known activity properties of the enzyme. In general, although the enzymes retain at least partial activity below their optimal extension temperatures, polymerase extension by the thermostable polymerases most often useful in the methods described herein (e.g., Vent Exo⁻ DNA polymerase, Tfl DNA polymerase, 9° Nm DNA polymerase, Tfu DNA polymerase, etc.) is performed at 65° C. to 75° C., preferably about 68-72° C.

As used herein, the term “aliquot” refers to a sample of an amplification reaction taken during the cycling regimen. An aliquot is less than the total volume of the reaction, and is preferably 0.1-30% in volume. In one embodiment of the invention, for each aliquot removed, an equal volume of reaction buffer containing reagents necessary for the reaction (e.g., buffer, salt, nucleotides, and polymerase enzyme) is introduced. The phrase “sampling a reaction mixture” refers to removing an aliquot of an amplification reaction mixture.

As used herein, the term “separating nucleic acid molecules” refers to the process of physically separating nucleic acid molecules in a sample or aliquot on the basis of size or charge. Electrophoretic separation is preferred, and capillary electrophoretic separation is most preferred.

### BRIEF DESCRIPTION OF THE FIGURES

FIG. 1 shows the results of experiments comparing the activity of a range of thermostable DNA polymerase enzymes in genetic profiling analysis as described herein. FIG. 1a shows a plot of Log₁₀ of the area under the curve versus cycle number for parallel assays performed with Vent Exo⁻™ (diamonds), 9° Nₘ (squares), Pfu (triangles), Taq (Xs), Tfu (asterisk) and Tgo (circle) DNA polymerases. FIG. 1b shows elctropherograms from cycle 30 of the assays shown in FIG. 1a.

FIG. 2 shows a comparison of Vent Exo⁻™ DNA polymerase to Tfu DNA polymerase in genetic profiling analyses. Diamonds show the amplification curve obtained with Vent Exo⁻™ DNA polymerase. Squares show the amplification curve obtained with Tfu DNA polymerase.

FIG. 3 shows comparison of multiplex analyses using 9° Nₘ and Vent Exo⁻™ DNA polymerases.

FIG. 4 shows a comparison of the performance of Tfl DNA polymerase versus Vent Exo⁻™ DNA polymerase in a multiplex genetic profiling assay. Squares show the amplification curve generated with Vent Exo⁻™, and the triangles show the curve generated with Tfl.

### DESCRIPTION

Described herein are thermostable nucleic acid polymerases that are well suited for genetic profiling methods, as well as genetic profiling methods that use those polymerases. The thermostable polymerases described are particularly well suited for genetic profiling methods involving the use of 5′ labeled primers, amplification of multiple templates in a single reaction (so called “multiplex reactions”), or both. Amplification and genetic or expression profiling processes are described herein below, as are thermostable DNA polymerases and the properties which render them well suited for the described processes.

Nucleic Acid Amplification:

Described herein are methods for analyzing nucleic acids in a sample. The methods described herein permit the detection of nucleic acids for, e.g., pathogen detection as well as the measurement of e.g., gene expression, both in a single individual or sample and between two or more individuals or samples. The methods rely on the ability of a nucleic acid amplification regimen, e.g., the polymerase chain reaction (PCR), to quantitatively produce copies of one or more sequences of interest in a sample in a number sufficient to detect those copies above the background of other sequences present in the sample.

In PCR, two oligonucleotide primers, a template and a thermostable nucleic acid polymerase are used. In the general PCR scheme, one of the oligonucleotide primers anneals to a template nucleic acid strand. The annealed primer is extended by the thermostable template-dependent nucleic acid polymerase, and that polymerization product has a sequence complementary to the second primer such that the polymerization product can serve as template for the extension of the second primer. The polymerization product is thermally denatured to separate the strands, and the pair of primers is annealed to the respective strands and extended. Because each extension product serves as the template for subsequent extension reactions, the target sequence is exponentially amplified.

The amount of amplification product produced in a reaction can reflect the amount of template present in the original sample. While PCR is a powerful tool in the quantitative analysis of gene expression, it is known in the art that quantitative amplification requires careful and sometimes elaborate controls in order to ensure that the amount of product detected accurately reflects the amount of target RNA or DNA present in the original sample. Quantitative PCR is described, for example by Reischl & Kochanowski, 1995; Mol. Biotechnol. 3:55-71, and Jung et al, 2000, Clin. Chem. Lab. Med. 38:833-836. The same is generally also true of other nucleic acid amplification approaches.

PCR amplification is only quantitative during the exponential phase of the amplification. After the exponential phase gives way to a plateau, the amount of product detected does not accurately reflect the amount of target sequence present in the initial sample. The exhaustion of primer and nucleotide supplies, the accumulation of phosphate and the accumulation of amplified products themselves all contribute to non-linearity between the amount of input nucleic acid and the amount of output PCR product after the exponential phase of the amplification. In addition, when two or more distinct targets are amplified, differences in the efficiency of amplification of one product versus another will result in inaccuracy in the relative quantitation of target molecules present in the original sample. Thus, in order to obtain meaningful results from quantitative PCR methods, it is important to be able to monitor not only the amount of product generated, but how and when that product was generated.

The methods as described herein overcome a number of the difficulties involved in quantitative nucleic acid amplification. For example, the methods described herein permit the determination of the amount of amplification product made and simultaneously provide a curve showing the manner in which the product was made. The methods thus permit the distinction between amplification signal arising from desired product and from artifacts, such as primer dimer formation or mispriming events.

Polymerases:

There are three major families of DNA polymerases, termed families A, B and C. The classification of a polymerase into one of these three families is based on structural similarity of a given polymerase to E. coli DNA polymerase I (Family A), II (Family B) or III (Family C). As examples, Family A DNA polymerases include, but are not limited to Klenow DNA polymerase, Thermus aquaticus DNA polymerase I (Taq polymerase) and bacteriophage T7 DNA polymerase; Family B DNA polymerases, formerly known as α-family polymerases (Braithwaite and Ito, 1991, Nuc. Acids Res. 19:4045), include, but are not limited to human α, δ and ε DNA polymerases, T4, RB69 and φ29 bacteriophage DNA polymerases, and Pyrococcus furiosus DNA polymerase (Pfu polymerase); and Family C DNA polymerases include, but are not limited to Bacillus subtilis DNA polymerase III, and E. coli DNA polymerase III α and ε subunits (listed as products of the dnaE and dnaQ genes, respectively, by Brathwaite and Ito, 1993, Nucleic Acids Res. 21: 787). An alignment of DNA polymerase protein sequences of each family across a broad spectrum of archaeal, bacterial, viral and eukaryotic organisms is presented in Braithwaite and Ito (1993, supra), which is incorporated herein by reference. Hopfner et al. also aligned the sequences of polymerases encoded by Thermococcus gorgonarius, Pyrococcus furiosus, Thermococcus litoralis, Methanococcus voltae, bacteriophage RB69, bacteriophage T4, and E. coli pol II and human pol δ. These alignments, considered with functional affects of mutations on members of a family permit the assignment of structure/function correlations to the various conserved domains of the polymerases.

Polymerases as described herein are advantageously employed in methods for the analysis of nucleic acids as described herein and in co-pending U.S. patent application Ser. No. 10/113,034, filed Apr. 1, 2002, the entirety of which is incorporated herein by reference.

All thermostable polymerases are not created equal with respect to the methods described herein. It has been discovered that several enzyme characteristics are important for determining whether a given enzyme is or is not suitable for the methods described herein. Because 5′ end labeling is a preferred labeling approach, it is preferred, for example, that the chosen polymerase enzyme have little or no 5′-3′ exonuclease activity. A number of wild-type or mutant enzymes are known to those skilled in the art that have little or no 5′-3′ exonuclease activity and would thus satisfy this criterion for a polymerase of use in the methods described herein.

Further, in order to optimize the sensitivity of an assay as described herein for the detection and quantitation of small amounts or low initial copy numbers of nucleic acid material, a polymerase useful in the methods described herein should have low 3′-5′ exonuclease (proofreading) activity, if any. While not wishing to be bound by any single mechanism, it is thought that low proofreading activity enhances sensitivity by increasing the processivity of the polymerase enzyme. An enzyme that is more processive than another incorporates more nucleotides per initiation event than the other and is therefore more efficient, if other parameters are similar. A more efficient enzyme produces more product per cycle and can therefore amplify smaller amounts of starting material to detectable levels more readily than a less efficient enzyme. Enzymes with low proofreading activity are known in the art and discussed herein above, as are domains which can be targeted if one desires to modify an existing enzyme with proofreading activity for a method described herein. It is preferred that the enzyme used in methods described herein be at least as processive as Vent Exo-™.

It has also been discovered, however, that when multiplex detection of target nucleic acids is desired, e.g., assays aimed at the detection of 5 or more, or, e.g., 10 or more target sequences in a single reaction, it can be helpful for the enzyme used to have proofreading activity. Again, without wishing to be bound by one specific mechanism, it is thought that in situations where there are a number of different primers present in a multiplex reaction, some proofreading activity can be of use in eliminating or reducing the incidence of mispriming events that may lead to artifacts. The benefit of increased multiplex ability provided by proofreading activity comes at the cost of somewhat reduced sensitivity, but the trade-off can be acceptable where it is more important to quickly gauge the presence and abundance of a number of different targets in a given sample than it is to measure small initial amounts of a single target. It is contemplated that for multiplex detection where one does not wish to sacrifice sensitivity, an enzyme with reduced, but not abolished proofreading activity can be useful. Thus, for example, in some embodiments, conservative exonuclease motif I mutants such as those described by Derbyshire et al. (supra) which have partial proofreading activity relative to wild-type counterparts may be advantageous in multiplex assays.

Finally, it has been discovered that strand displacement activity of the polymerase is an important parameter for the methods described herein. Polymerases having at least some strand displacement activity perform better than those without such activity. Without wishing to be confined to any single mechanism, it is thought that the strand displacement activity aids in the displacement, by the advancing polymerase, of nucleic acids non-specifically hybridized to a template strand. Such activity would tend to increase the efficiency of the polymerization, particularly when there are a relatively large number of different primers present, e.g., as in a multiplex reaction. In addition, strand displacement activity correlates well with reduced background in amplification assays. Where desired or necessary, the measurement of strand displacement activity can be performed by one of skill in the art using methods known in the art or methods described herein below.

Strand Displacement Activity:

Strand displacement activity is measured, for example, using the methods taught by Kong et al., 1993, J. Biol. Chem. 268: 1965-1975. Briefly, a ³²P-end-labeled primer is annealed to a single-stranded template, e.g., M13 mp18 single stranded DNA, and an unlabeled primer is annealed downstream of the labeled primer (distance can vary, but is conveniently about 80 nucleotides downstream) such that there is a gap between the 3′ end of the labeled primer and the 5′ end of the downstream primer. Salt, buffer, co-factor and nucleoside precursor conditions should be those known to be optimal for the polymerase to be tested. Polymerase is then added and polymerization is performed at the optimal temperature for the enzyme. Aliquots are removed as a function of time, added to stop solution containing formamide plus 0.37% EDTA (pH 7.0) and incubated on ice until all samples are collected. Samples are then separated on a denaturing polyacrylamide gel (e.g., 6% acrylamide, 6 M urea) in Tris/borate/EDTA buffer and visualized by autoradiography. For quantitation, densitometry or phosphorimaging can be performed. The strand displacement activity of a number of polymerase enzymes, both thermostable and non-thermostable, is known in the art. See, for example, the New England Biolabs DNA Polymerase Technical Bulletin (on the world wide web at www.neb.com/nebecomm/tech_reference/polymerases/polymerases_from_neb.asp), which notes the 3′-5′ exonuclease and strand displacement activities for 11 different thermostable DNA polymerases including Vent™, Vent Exo-™, Deep Vent™, Deep Vent Exo-™ and 9° Nₘ, among others. Vent Exo-™ is listed in that reference as having better strand displacement activity than wild-type Vent™, and 9° Nₘ is listed as having similar strand displacement activity to VentExo⁻™.

Exonuclease Activities:

Nucleic acid polymerases of use in the methods described herein are preferably 5′-3′ exonuclease deficient thermostable nucleic acid polymerases. Also, in particular embodiments, it is preferred that the chosen polymerase either specifically has 3′-5′ exonuclease (proofreading) activity or has low or absent 3′-5′ exonucleases activity. The structures of a variety of DNA polymerases are well known, as are structure/function correlations between particular domains of such polymerases.

Nucleic acid polymerases, particularly the thermostable polymerases encoded by thermophilic archaea tend to have conserved structural domains that correlate with specific functions. For example, exonuclease activity (5′-3′ and 3′-5′) and nucleotide discrimination function are localized to specific domains of the polymerase. Such domain structures and their respective functions are described, for example, in Hopfner et al., 1999, Proc. Natl. Acad. Sci. U.S.A. 96: 3600-3605, Kong et al., 1993, J. Biol. Chem. 268: 1965-1975, Ito & Braithwaite, 1991, Nucleic Acids Res. 19: 4045-4057, Braithwaite & Ito, 1993, Nucl. Acids Res. 21: 787-802, Gardner & Jack, 1999, Nucl. Acids Res. 27: 2545-2553, Takagi et al., 1997, Appl. Environ. Microbiol. 63: 4504-4510, Southworth et al., 1996, Proc. Natl. Acad. Sci. U.S.A. 93: 5281-5285, each of which is incorporated herein by reference.

At least three 3′-5′ exonucleases motifs have been identified through sequence alignment/comparison and mutagenesis studies. These are described in Blanco et al, 1991, Gene 100: 27-38 and Morrison et al., 1991, Proc. Natl. Acad. Sci. U.S.A. 88: 9473-9477, and reviewed in, e.g., Derbyshire et al., 1995, Meth. Enzymol. 262: 363-388. In one motif, for example, the motif termed 3′-5′ exo motif I, also known as the DXE motif, exonuclease activity is correlated with a conserved FDIET (SEQ ID NO: 3) amino acid sequence motif. The FDIET sequence motif is shared, for example, by Vent™ DNA polymerase, Pfu DNA polymerase, and Deep Vent DNA polymerase (see, e.g., Vemori et al., 1993, Nucl. Acids Res. 21: 259-265), as well as, e.g., Thermococcus barossii DNA polymerase (see U.S. Pat. No. 5,882,904), among others. Substitution of either or both of the conserved Asp (D) or Glu (E) residues in the motif (e.g., modification of FDIET to FAIAT (SEQ ID NO: 4)) with Ala (A) abolishes 3′-5′ exonuclease activity in these polymerases, while conservative mutations result in variable reductions in proofreading activity (Derbyshire et al., supra). The FAIAT mutation is the exo⁻ mutation present in Vent Exo-™ polymerase (SEQ ID NO: 2) described herein. Southworth et al. report that similar mutations in the Asp-Xaa-Glu (DXE) motif I sequence of the Thermococcus sp. 9° N-7 DNA polymerase have similar effects, either abolishing or dramatically reducing (reduction by more than 95% relative to wild type) 3′-5′ exonucleases activity while maintaining polymerase activity (Southworth et al., 1996, Proc. Natl. Acad. Sci. U.S.A. 93: 5281-5285; Rodriquez et al., 2000, J. Mol. Biol., 299: 447-462). The mutant 9° Nₘ™ polymerase is 3′-5′ exonuclease deficient by virtue of DXE motif mutation (see U.S. Pat. No. 5,756,334). Where necessary for the methods described herein, the exonucleases activity of a given DNA polymerase enzyme can be assayed according to the method described by Southworth et al., supra. The reference describes several different assays in which different templates or sources of enzyme (e.g., purified versus crude lysate) are used. Any of these assays can be used, but for the avoidance of doubt, Assay 3 is preferred. The assay uses a single-stranded 32-mer labeled at the 5′ end using γ-³²P-dATP and T4 polynucleotide kinase. This labeled DNA substrate (80 nM) is incubated in Vent™ buffer (New England Biolabs; alternatively, buffer conventionally used with a given enzyme being assayed can be used) at 72° C. for up to 30 minutes and then mixed with DNA sequencing stop dye (New England Biolabs). Samples are heated at 90° C. to denature and electrophoresed through a 20% urea denaturing gel, followed by drying and autoradiography of the gel.

Non-limiting examples of polymerases satisfying the characteristics of having strand displacement activity with low 5′-3′ and 3′-5′ exonuclease activity include Vent Exo⁻™ and 9° Nₘ. Each of these enzymes works well in the methods described herein. It is noted that Tfl DNA polymerase (initially isolated from Thermus flavus, available, e.g., from Promega; see Kaledin et al., 1981, Biokhimiia [Isolation and properties of DNA-polymerase from the extreme thermophilic bacterium Thermus flavus] 46:1576-84, and Akhmetzjanov and Vakhitov, 1992, Nucl. Acids Res. 20: 5839; see, e.g., GenBank Accession No. P30313) has strand displacement activity and lacks 3′-5′ exonuclease activity, but has 5′-3′ exonuclease activity yet works to a degree similar to Vent Exo⁻™ in methods described herein. Thus, while it retains 5′-3′ exonuclease activity, the Tfl DNA polymerase is suitable for the methods described. Without wishing to be bound by any one mechanism, it is possible that the high processivity of the Tfl enzyme counteracts any negative impact of the 5′-3′ exonuclease activity.

As a further specific reference, Tfu DNA polymerase (see, e.g., GenBank Accession Nos. P74918 and CAA93738) has strand displacement activity, lacks 5′-3′ exonuclease activity, yet has 3′ to 5′ exonucleases activity. This enzyme works in the methods described herein, although not as well as, for example Vent Exo⁻™. The Tfu enzyme, with its 3′-5′ exonuclease activity, is anticipated to be well suited for multiplex assay formats, which, as discussed herein, have been found to benefit proofreading activity.

For comparison, enzymes such as Taq polymerase, which lack strand displacement activity, are not favored for the methods described herein.

Amplification Monitoring:

In one aspect, the invention provides a method of monitoring the amplification of a single target nucleic acid from one sample. This and other aspects described are suited for monitoring the amplification of polynucleotide sequences from samples containing DNA, RNA, or a mixture of these, but will most often find application in measuring or comparing RNA transcript levels in a sample. For the measurement of RNA transcripts, a reverse-transcription step is required. Reverse-transcription methods are well known in the art. Depending upon the desired assay, reverse-transcription can be performed using a non-specific primer (e.g., an oligo-dT-containing primer or a collection of random primers), or reverse-transcription can be performed using a primer specific for a gene of interest.

In this aspect, a nucleic acid sample is subjected to a PCR amplification regimen comprising at least two cycles of thermal denaturation, primer annealing and primer extension. The amplification regimen will preferably comprise 2 to 35 cycles, more preferably 10 to 30 cycles, more preferably 15 to 25 cycles. The regimen uses a pair of oligonucleotide primers that result in the specific amplification of a single sequence of interest if that sequence is present in the sample. One of the oligonucleotide primers is fluorescently labeled, such that each double-stranded amplification product will bear a detectable fluorescent marker.

During the cycling regimen, following at least one of the cycles of denaturation, primer annealing and primer extension in this aspect of the invention, a sample or aliquot of the reaction is withdrawn from the tube or reaction vessel, and nucleic acids in the aliquot are separated and detected. The separation and detection are preferably performed concurrently with the cycling regimen, such that a curve representing product abundance as a function of cycle number is generated while the cycling occurs. Depending upon the separation technology used (e.g., capillary electrophoresis) and the number and size of species to be separated in a given reaction, the separation will most often require on the order of 1-120 minutes per aliquot.

In the manner described above, one can monitor the amplification reaction in real time, and the correlation between target product abundance and amount of target sequence in the original sample permits accurate and rapid target quantitation. Aliquot removal (also referred to herein as “sampling”), nucleic acid separation, and detection can be performed as also described herein.

At the beginning of the PCR amplification reaction, the amount of PCR product is below the detection limit of most instruments and no quantitative difference can be observed. For the detection of rare gene transcripts which are normally present at the level of several copies per cell, monitoring PCR products at very late stages will be necessary. Typically, detection of these genes will be difficult because the reaction is stopped before those rare transcripts are amplified to a detectable level. The middle section of the amplification curve, when the signal arises above the detection limit and enters a logarithmic phase, constitutes the best signal for detecting quantitative differences in gene expression. However, due to the exponential nature of the reaction, this phase is relatively short and lasts only a few cycles before the reaction goes into a later stationary phase. In this later stationary phase of PCR amplification, accumulation of PCR products are saturated due to factors such as lack of additional substrates, lack of polymerase, inhibition of polymerase activity by the product, or a combination thereof. The stationary phase provides little opportunity for detecting quantitative differences in gene expression. Therefore, methods that quantify PCR product after a predetermined number of cycles can quantitate only genes that happen to be in the logarithmic phase of the amplification after that number of cycles, and would thus miss those genes that are only differentially detected either earlier or later in the amplification process.

The sampling approach described herein instead defines a complete amplification curve for each individual amplified fragment. Moreover, it provides a quantitative basis for measuring expression differences. In this method of target sequence quantitation, the experimentally defined parameter “Cₜ” refers to the cycle number at which the signal generated from a quantitative amplification reaction first rises above a “threshold”, i.e., where there is the first reliable detection of amplification of a target nucleic acid sequence. “Reliable” means that the signal reflects a detectable level of amplified product during the amplification regimen. Cₜ generally correlates with starting quantity of an unknown amount of a target nucleic acid, i.e., lower amounts of target result in later Cₜ. Cₜ is linked to the initial copy number or concentration of starting DNA by a simple mathematical equation:
Log(copy number)=aCₜ+b, where a and b are constants.

Therefore, by measuring Cₜ for the fragments of the same gene originating from two different samples, the original relative concentration of this gene in these samples can be easily evaluated.

One of the most interesting features of the PCR amplification is the ability to combine amplification of several target sequences in a single reaction which can provide a significant savings in time and cost of PCR assays. There are methods available that permit the co-amplification of multiple different polynucleotide sequences in a single amplification reaction. See e.g., Markoulatos et al. 2002, J. Clin. Lab. Anal. 16:47-51 and Broude et al., 2001, Antisense Nucleic Acid Drug Dev. 11:327-332.

In another aspect, the methods described herein improve upon the multiplex quantitation of target sequences. Specifically, the methods described herein permit the monitoring of the amplification of more than one polynucleotide sequence in a sample. The methods described generate an amplification profile for two or more sequences that permits one to correlate signal strength during the logarithmic phase of the amplification to the relative amount of those sequences present in the original sample.

In this aspect, a set of oligonucleotide primers is used, and the set is comprised of pairs of oligonucleotide amplification primers specific for the two or more genes of interest. One primer of each pair of primers is preferably fluorescently labeled with a fluorescent marker. In one embodiment, the different primers can be labeled with the same fluorescent marker. In that instance, the primers for the different amplification products are selected such that the sizes of the amplified products are distinct. As used in this context, “distinct” refers to a difference in sequence length that can be distinguished using the selected separation technology; amplified products differing by as little as a single nucleotide are distinct as long as the separation technology (e.g., capillary electrophoresis—see below) is capable of resolving that difference. PCR products useful according to the invention will generally be at least 50 bp in length, and can be as long as about 5,000 bp. Most often, PCR fragment lengths useful according to the methods described herein will be on the order of 50-1000 bp in length, preferably 50-500 bp.

In another embodiment, one member of each pair of amplification primers is fluorescently labeled with a fluorophore that is spectrally distinguishable from the fluorophores labeling a member of each other pair of primers. In this embodiment, the sizes of the expected amplification products can be, but need not be distinct. It is assumed that spectrally distinguishable fluorophores used in a given reaction are selected such that they do not quench or engage in energy transfer with other fluorophores in the same reaction.

In an embodiment that further increases the capacity for simultaneous detection of different sequences in a single sample, the pairs of oligonucleotides are selected such that each pair generates either a distinctly sized fragment or is labeled with a distinct fluorophore or both. In this embodiment, different products that are the same size will have spectrally distinct fluorophores, and different products that are labeled with the same fluorophore will have distinct sizes.

In this aspect of the invention, once the desired combination of label and expected product sizes is selected, the nucleic acid sample is contacted with the set of pairs of primers, in an amplification reaction mixture comprising a nucleic acid polymerase, nucleotides and necessary buffer. The reaction mixture is then subjected to an amplification regimen comprising, e.g., iterative cycles of thermal denaturing, primer annealing, and primer extension. During this regimen, aliquots of the reaction are removed as described herein (see “Sampling” section, below) and the nucleic acids in the aliquot are separated as also described herein below.

The separation and detection steps permits the identification of amplified products by their size, fluorescent marker, or both, depending upon the combination of expected product sizes and spectrally distinguishable markers used. Thus, when the products bear the same marker, the separation will generate a “ladder” of fragments comprising the same label, and fragment size will distinguish one product from another. When the products bear different labels, the separation will generate bands comprising each different label that identify the amplified products. Finally, when the products are selected to be of distinct sizes and be distinctly labeled, the products are uniquely identified by the wavelength of fluorescence and the size of the fragment.

Aside from identifying the products, the detection step determines the abundance of each product present at each cycle sampled. This abundance can be plotted for each fragment to generate a fragment-specific amplification profile which can be used to extrapolate the relative abundance of the sequence represented by each detected fragment in the original sample.

In another aspect of the methods described herein, the amplification of a set of nucleic acid fragments present in a single sample can be monitored by using a set of pairs of forward and reverse oligonucleotide primers specific for each nucleic acid fragment of interest in the set of nucleic acid fragments. Primer pairs are selected so that the amplification product of each pair will be distinctly sized relative to other amplification products in the same reaction. Amplification is performed in the presence of a fluorescent dye (e.g., SYBR-Green; Molecular Probes Inc., Eugene, Oreg.) that binds only double-stranded DNA. During the amplification regimen, aliquots of the reaction are removed and the nucleic acids in the aliquot are separated as described herein. In this aspect of the invention, as in the others, the separation process is performed concurrently with the cycling reaction, and amplified double-stranded nucleic acids are detected by fluorescence and size. The abundance of each amplified fragment is identified by fluorescence intensity of the bound dye. The distinct sizes of the amplification products permit one to monitor the amplification of each nucleic acid fragment of interest throughout the amplification reaction, thereby generating an amplification curve for each nucleic acid fragment of interest. The curve indicates at which cycle each member of the set of nucleic acid fragments was in the logarithmic phase of amplification, thereby permitting extrapolation of the relative abundance of each member of the set in the original sample.

Separation of Nucleic Acids

Separation of nucleic acids according to the methods described herein can be achieved by any means suitable for separation of nucleic acids, including, for example, electrophoresis, HPLC or mass spectrometry. Separation is preferably performed by capillary electrophoresis (CE).

CE is an efficient analytical separation technique for the analysis of minute amounts of sample. CE separations are performed in a narrow diameter capillary tube, which is filled with an electrically conductive medium termed the “carrier electrolyte.” An electric field is applied between the two ends of the capillary tube, and species in the sample move from one electrode toward the other electrode at a rate which is dependent on the electrophoretic mobility of each species, as well as on the rate of fluid movement in the tube. CE may be performed using gels or liquids, such as buffers, in the capillary. In one liquid mode, known as “free zone electrophoresis,” separations are based on differences in the free solution mobility of sample species. In another liquid mode, micelles are used to effect separations based on differences in hydrophobicity. This is known as Micellar Electrokinetic Capillary Chromatography (MECC).

CE separates nucleic acid molecules on the basis of charge, which effectively results in their separation by size or number of nucleotides. When a number of fragments are produced, they will pass the fluorescence detector near the end of the capillary in ascending order of size. That is, smaller fragments will migrate ahead of larger ones and be detected first.

CE offers significant advantages of over conventional electrophoresis, primarily in the speed of separation, small size of the required sample (on the order of 1-50 nl), and high resolution. For example, separation speeds using CE can be 10 to 20 times faster than conventional gel electrophoresis, and no post-run staining is necessary. CE provides high resolution, separating molecules in the range of about 10-1,000 base pairs differing by as little as a single base pair. High resolution is possible in part because the large surface area of the capillary efficiently dissipates heat, permitting the use of high voltages. In addition, band broadening is minimized due to the narrow inner diameter of the capillary. In free-zone electrophoresis, the phenomenon of electroosmosis, or electroosmotic flow (EOF) occurs. This is a bulk flow of liquid that affects all of the sample molecules regardless of charge. Under certain conditions EOF can contribute to improved resolution and separation speed in free-zone CE.

CE can be performed by methods well known in the art, for example, as disclosed in U.S. Pat. Nos. 6,217,731; 6,001,230; and 5,963,456, which are incorporated herein by reference. High throughput CE equipment is available commercially, for example, the HTS9610 High Throughput Analysis System and SCE 9610 fully automated 96-capillary electrophoresis genetic analysis system from Spectrumedix Corporation (State College, Pa.). Others include the P/ACE 5000 series from Beckman Instruments Inc (Fullerton, Calif.) and the ABI PRISM 3100 genetic analyzer (Applied Biosystems, Foster City, Calif.). Each of these devices comprises a fluorescence detector that monitors the emission of light by molecules in the sample near the end of the CE column. The standard fluorescence detectors can distinguish numerous different wavelengths of fluorescence emission, providing the ability to detect multiple fluorescently labeled species in a single CE run from an amplification sample.

Another means of increasing the throughput of the CE separation is to use a plurality of capillaries, or preferably an array of capillaries. Capillary Array Electrophoresis (CAE) devices have been developed with 96 capillary capacity (e.g., the MegaBACE instrument from Molecular Dynamics) and higher, up to and including even 1000 capillaries. In order to avoid problems with the detection of fluorescence from DNA caused by light scattering between the closely juxtaposed multiple capillaries, a confocal fluorescence scanner can be used (Quesada et al., 1991, Biotechniques 10:616-25).

The apparatus for separation (and detection) can be separate from or integrated with the apparatus used for thermal cycling and sampling. While it is not necessary, it is preferred that the separation apparatus is integral with the thermal cycling and sampling apparatus. In one embodiment, this apparatus is modular, comprising a thermal cycling module and a separation/detection module, with a robotic sampler that withdraws sample from the thermal cycling reaction and places it into the separation/detection apparatus.

### EXAMPLES

Sequences for Primers and Details for SARS Experimental Target Sequences are Provided in Appendix 2

### Example 1

### Comparison of Various DNA Polymerases in Profiling Assays Performed Using a DNA Template

In order to evaluate which thermostable DNA polymerases are suitable for the genetic profiling methods described herein, an artificial DNA template, termed VS85 was amplified using the following thermostable DNA polymerase enzymes: Vent Exo⁻™ (New England Biolabs); 9° Nₘ (New England Biolabs); Pfu (Stratagene); Taq (Qiagen), Tfu (Promega) and Tgo (Roche).

Method: 10 ng of VS85 DNA template was PCR amplified using either Vent (exo-), 9oNm, Pfu (Stratagene), Taq (Qiagen), Tfu (Promega) or Tgo (Roche) in IX reaction buffer as supplied by the manufacturer with the addition of 200 uM dNTP, 0.02% DMSO, 20% Q solution (Stratagene), and 1 uM VS85-specific primers (FAM-LHA and pcDNA3L). PCR protocol consisted of an initial denaturation step of 95° C. for 5 min, followed by 30 cycles of 95° C./30 sec, 56° C./30 sec, 72° C./1 min. Three ul aliquots were collected from cycles 10 to 30 at the end of each cycle, transferred to 7 ul formamide containing 0.3 ul ROX1000 DNA standards (Bioventures), denatured at 95° C. for 5 min and separated by capillary electrophoresis on the ABI 3100 Genetic Analyzer using POP-4 polymer at 15 kV for 35 min.

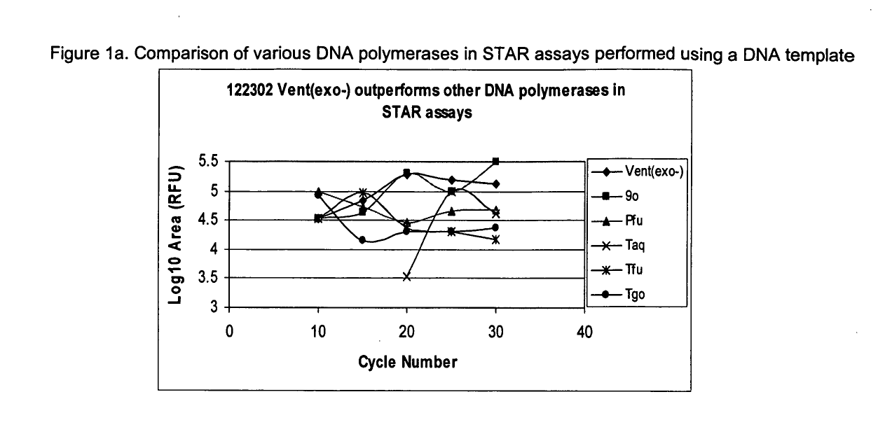

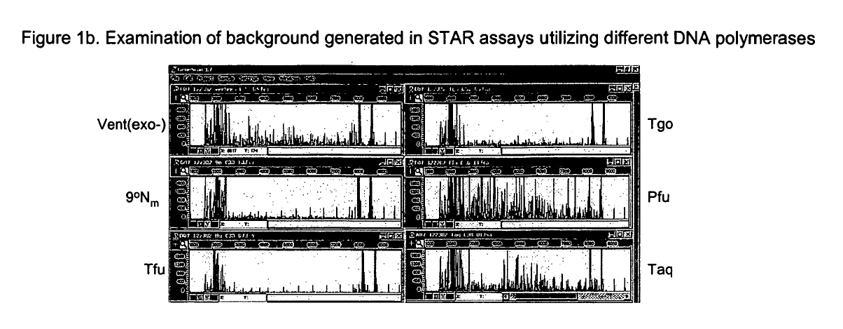

Results are shown in FIG. 1a. Vent Exo-™ performed better than or equal to the other assessed DNA polymerases in this assay. Taq, Tgo and Pfu polymerases were significantly less effective than Vent Exo⁻™ or, for example, 9° Nₘ polymerase.

Relative levels of background amplification signal were examined at cycle 30 for each DNA polymerase tested. The electrophorograms are shown in FIG. 1b. Peaks that appear at the left of the respective electropherograms represent unincorporated primers while the peaks at the right of each electrophorogam represent the amplification products of VS85. While all DNA polymerases worked to varying degrees, levels of background amplification are lowest with polymerases showing high strand displacement activity. Note that Tgo also shows very low background; however, specific amplification was very poor (see FIG. 1a).

### Example 2

### Comparison of Vent Exo-™ DNA Polymerase to Tfu DNA Polymerase in Multiplex Expression Profiling Assays

Vent Exo-™ DNA polymerase is deficient in both 5′-3′ and 3′-5′ exonucleases activities. The following experiments were performed in order to compare this polymerase to another selected thermostable DNA polymerase enzyme in expression profiling assays using artificial test transcripts spiked into a sample of rat brain total RNA.

Method: For reverse transcription (RT), 1.75 ug total rat brain RNA spiked with 100 ug of each of two artificial transcripts, VS32 and VS85 and sequence-tagged oligo(dT) primer (ST1dT15VN, 1.25 uM) was added to 58% glycerol, heated at 70° C. for 10 min, then put on ice for 2 min. Buffer (final concentrations: 50 mM Tris-HCl, pH 8.3, 75 mM KCl, 3 mM MgCl2, 0.01M DTT, 0.8 mM dNTP, 0.2 mg/ml BSA, 20% trehalose), 3.2 U/μl of Superscript II RNase H-Reverse Transcriptase (SSRTII; Invitrogen) and 1 U/μl of RNAsin (Ambion) were added and reverse transcribed at 45° C. for 20 min, followed by denaturation at 75° C. A second round of RT at 48° C. for 20 min was initiated with the addition of 50 U SSRTII followed by a third round of RT at 52° C. for 20 min after a 2 min 80° C. denaturation step. Samples were alkaline treated with 0.04 M NaOH (final concentration) for 15 min at 65° C., followed by addition of Tris pH 7.5 to a final concentration of 70 mM. Resultant cDNAs were purified using the QIAquick Gel Extraction Kit (Qiagen) as per manufacturers instructions, except that 360 μl of QG buffer was added to each sample.

For second strand synthesis, purified cDNA in 40 mM Tris (pH 7.5), 20 mM MgCl₂, 50 mM NaCl, 0.2 mM dNTPs was heat denatured at 95° C. for 1 min followed by addition of 1.6 uM second strand primer (UT2-HA) and continued denaturation at 95° C. for 4 min. The reaction was ramped to 37° C., 0.5 U/μl Sequenase™ DNA polymerase (USB, Cleveland, Ohio) was added and incubated for 1 hr. DNA was purified using the QIAquick™ Gel Extraction Kit as above. 0.5 uM primers (FAM-ST1 and UT2) were added and PCR amplification using Vent(exo-)™ proceeded in 10 mM KCl, 10 mM (NH₄)₂SO₄, 20 mM Tris-HCl (pH 8.8), 2 mM MgSO₄, 0.1% Triton X-100, 0.2 mM dNTPs, 20% Q solution (Stratagene), 2% DMSO, 2 U Vent(Exo-)™ DNA polymerase overlaid with mineral oil. PCR amplification using Tfu DNA polymerase proceeded in 10 mM Tris pH 9.0, 50 mM KCl, 1.5 mM MgCl₂, 0.1% TX100, 0.2 mg/ml BSA, 2U Tfu DNA polymerase overlaid with mineral oil. PCR protocol: 95° C. for 5 min followed by 43 cycles of 95° C./30 sec, 62° C./30 sec, 72° C./1 min. Three μl aliquots were collected from cycles 20 to 43 at the end of each cycle.

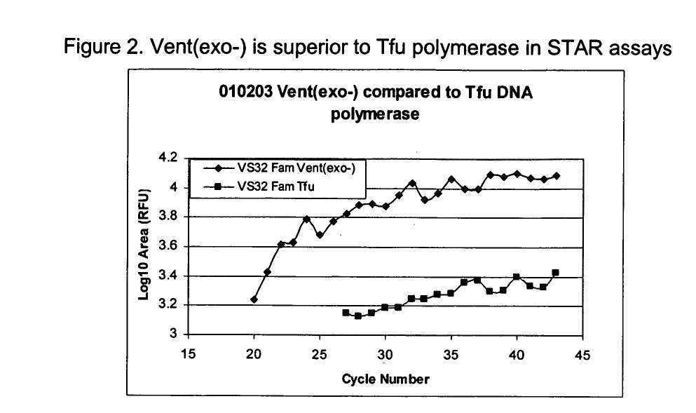

Results for this assay are shown in FIG. 2, in which the signal from the transcript VS32 is shown for the assay using Vent Exo-™ versus the assay using Tfu DNA polymerase. The data show that while both enzymes gave detectable signal for this transcript, the Vent Exo⁻™ performed better than the Tfu DNA polymerase, with detection at a lower threshold cycle number and with a higher overall signal.

### Example 3

### Multiplex Amplification of SARS Targets from DNA Templates Using 9° Nₘ and Vent Exo⁻™ DNA Polymerase

The performance of Vent Exo-™ and 9° Nₘ DNA polymerases was examined in a five way multiplex assay.

Method: Five SARS targets were multiplex amplified from 100 ng of SARS DNA library. Samples were PCR amplified using 2U of Vent(exo-) or 9° Nₘ in 10 mM KCl 10 mM (NH₄)₂SO₄, 20 mM Tris pH 8.8, 2 mM MgSO₄, 0.1% TX100, 200 uM dNTP, 20% Q solution (Qiagen), 0.02% DMSO, 0.5 uM gene-specific primers directed toward the N (138base), Rep1A (206base) M (248base), Rep1B (403base) and S (465base) gene products. PCR protocol consisted of an initial denaturation step of 95° C. for 5 min, followed by 40 cycles of 95° C./30 sec, 62° C./30 sec, 72° C./1 min. Three ul aliquots were collected at the end of cycle 40, transferred to 7 ul formamide containing 0.3 ul ROX1000 DNA standards (Bioventures), denatured at 95° C. for 5 min and separated by capillary electrophoresis on the ABI 3100 Genetic Analyzer using POP-4 polymer at 15 kV for 35 min.

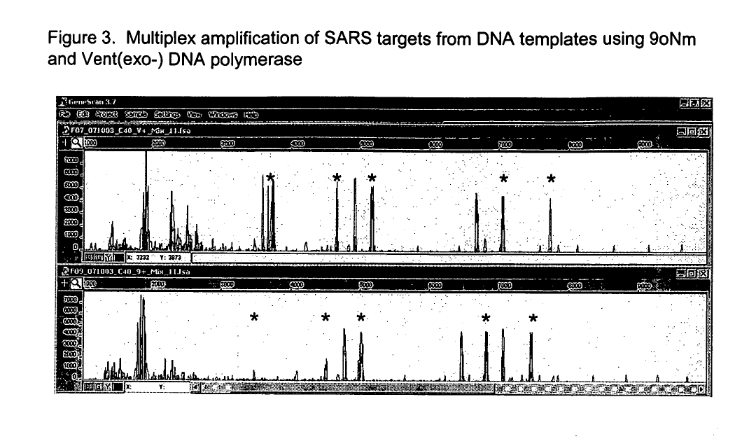

Results are shown in FIG. 3. Asterisks represent the 5 amplified species detected.

### Example 4

### Comparison of Tfl DNA Polymerase to Vent Exo⁻™ DNA Polymerase in Genetic Profiling Assays

In order to compare the usefulness of Tfl DNA polymerase with that of Vent Exo⁻™ DNA polymerase in profiling assays, a separate multiplex assay was performed using each of these polymerases.

Method: Multiplex RT-PCR was performed using 100 ng total RNA from rat brain (Stratagene) which was combined with buffer (50 Mm Tris-HCL pH (8.3), 75 mM KCl and 3 mM MgCl₂), 200 uM dNTP, 200 nM primers, Transcriptor RT (Roche-Applied Science) and either 0.1U Tfl DNA Polymerase (Promega) or 2 U Vent(exo-) (New England Biolabs). Reverse transcription using Transcriptor was run at 45° C. for 45 minutes prior to touchdown PCR which included thermal cycling for 3 cycles at (94° C./30 sec, 65° C./30 sec, 72° C./30 sec), 3 cycles at (94° C./30 sec, 62.5° C./30 sec, 72° C./30 sec), 3 cycles at (94° C./30 sec, 60.0° C./30 sec, 72° C./30 sec) and 25 cycles at (94° C./30 sec, 57° C./30 sec, 72° C./30 sec). Aliquots of the multiplex reaction were sampled every two cycles from cycle number 13 to 33 with an initial aliquot taken at cycle ten. The sample (2 ul) was combined with 8 ul Hi-Di Formamide (Applied Biosystems), 0.3 ul ROX Standards(BioVenture), denatured at 95° C./5 min and separated by capillary electrophoresis on the ABI 3730x1 DNA Analyzer using POP-7 polymer at 15 kV for 35 min.

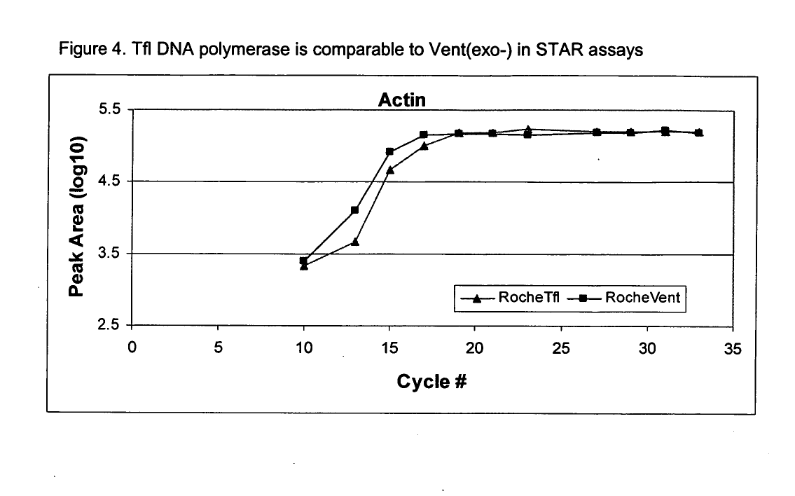

Results are shown in FIG. 4. The Tfl polymerase was found to perform comparably to Vent Exo⁻™.

1. A method for the analysis of a nucleic acid, the method comprising:
I) providing a reaction mixture by contacting a nucleic acid template with an oligonucleotide primer and a thermostable DNA polymerase that is at least as processive as VentExo⁻™ DNA polymerase, under conditions that permit the hybridization of said primer to said template, such that said nucleic acid template and said oligonucleotide primer are hybridized;
II) performing upon said reaction mixture at least two cycles of:
a) permitting the template-dependent extension of said primer by said DNA polymerase;
b) sampling said reaction mixture;
c) thermally separating the nucleic acid strands in said reaction mixture; and
d) permitting the hybridization of said primer to a said nucleic acid template;
II) separating the nucleic acid molecules in the sample taken at step (b);
III) detecting nucleic acid molecules separated in step (II), whereby said nucleic acid template is analyzed.

2. The method of claim 1 wherein said separating comprises capillary electrophoresis.

3. The method of claim 1 wherein said DNA polymerase has strand displacement activity, measured at optimal polymerizing temperature for said DNA polymerase, that is at least as great as the strand displacement activity of Vent Exo⁻™ DNA polymerase measured at 72° C.

4. The method of claim 1 wherein said DNA polymerase is 5′-3′ exonuclease-deficient.

5. The method of claim 1 wherein said DNA polymerase is 3′-5′ exonucleases deficient.

6. The method of claim 4 wherein said DNA polymerase is also 3′-5′ exonucleases deficient.

7. The method of claim 1 wherein said polymerase is selected from the group consisting of Vent Exo⁻™ DNA polymerase, Tfl DNA polymerase, 9° Nₘ DNA polymerase, and Tfu DNA polymerase.

8. The method of claim 1 wherein said reaction mixture of step (I) is a PCR reaction mixture that comprises at least 5 different template nucleic acid sequences and corresponding different PCR primer pairs specific for each of said at least 5 said different template nucleic acid sequences, and wherein said DNA polymerase has 3′ to 5′ exonuclease activity.

9. The method of claim 1 wherein said reaction mixture of step (I) is a PCR reaction mixture that comprises at least 10 different template nucleic acid sequences and corresponding different PCR primer pairs specific for each of said at least 10 said different template nucleic acid sequences, and wherein said DNA polymerase has 3′ to 5′ exonuclease activity.

10. A method for the analysis of a nucleic acid, the method comprising:
I) providing a reaction mixture by contacting a nucleic acid template with an oligonucleotide primer and a 5′-3′ exonuclease-deficient, thermostable DNA polymerase having strand displacement activity, under conditions that permit the hybridization of said primer to said template, such that said nucleic acid template and said oligonucleotide primer are hybridized;
II) performing upon said reaction mixture at least two cycles of:
a) permitting the template-dependent extension of said primer by said DNA polymerase;
b) sampling said reaction mixture;
c) thermally separating the nucleic acid strands in said reaction mixture; and
d) permitting the hybridization of said primer to a said nucleic acid template;
II) separating the nucleic acid molecules in the sample taken at step (b);
III) detecting nucleic acid molecules separated in step (II), whereby said nucleic acid template is analyzed.

11. The method of claim 10 wherein said separating comprises capillary electrophoresis.

12. The method of claim 10 wherein said detecting further comprises plotting a value for a nucleic acid molecule detected in a sample of step II(b).

13. The method of claim 10 wherein said reaction mixture is a PCR reaction mixture.

14. The method of claim 10 wherein said strand displacement activity, measured at optimal polymerizing temperature for said DNA polymerase, is at least as great as the strand displacement activity of Vent Exo⁻™ DNA polymerase measured at 72° C.

15. The method of claim 10 wherein said 5′-3′ exonuclease-deficient polymerase is also 3′-5′ exonuclease deficient.

16. The method of claim 10 wherein said polymerase is selected from the group consisting of Vent Exo⁻™ DNA polymerase, 9° Nₘ DNA polymerase, and Tfu DNA polymerase.

17. The method of claim 10 wherein a said oligonucleotide primer is 5′ labeled.

18. The method of claim 17 wherein said label is a fluorescent label.

19. The method of claim 10 wherein said detecting comprises measuring fluorescence.

20. The method of claim 10 wherein said polymerase is at least as processive as Vent Exo⁻™ DNA polymerase.

21. The method of claim 10 wherein said reaction mixture of step (I) is a PCR reaction mixture that comprises at least 5 different template nucleic acid sequences and corresponding different PCR primer pairs specific for each of said at least 5 said different template nucleic acid sequences, and wherein said 5′-3′ exonuclease-deficient, thermostable DNA polymerase has 3′ to 5′ exonuclease activity.

22. The method of claim 21 wherein said polymerase is Tfu DNA polymerase.

23. The method of claim 10 wherein said reaction mixture of step (I) is a PCR reaction mixture that comprises at least 10 different template nucleic acid sequences and corresponding different PCR primer pairs specific for each of said at least 10 said different template nucleic acid sequences, and wherein said 5′-3′ exonuclease-deficient, thermostable DNA polymerase has 3′ to 5′ exonuclease activity.

24. The method of claim 23 wherein said polymerase is Tfu DNA polymerase.

25. A method for the analysis of a nucleic acid, the method comprising:
I) providing a reaction mixture by contacting a nucleic acid template with an oligonucleotide primer and a 3′-5′ exonuclease-deficient, thermostable DNA polymerase having strand displacement activity, under conditions that permit the hybridization of said primer to said template, such that said nucleic acid template and said oligonucleotide primer are hybridized;
II) performing upon said reaction mixture at least two cycles of:
a) permitting the template-dependent extension of said primer by said DNA polymerase;
b) sampling said reaction mixture;
c) thermally separating the nucleic acid strands in said reaction mixture; and
d) permitting the hybridization of said primer to a said nucleic acid template;
II) separating the nucleic acid molecules in the sample taken at step (b);
III) detecting nucleic acid molecules separated in step (II), whereby said nucleic acid template is analyzed.

26. The method of claim 25 wherein said DNA polymerase is selected from the group consisting of Vent Exo⁻™ DNA polymerase, Tfl DNA polymerase, and 9° Nₘ DNA polymerase.

27. The method of claim 25 wherein said separating comprises capillary electrophoresis.

28. The method of claim 25 wherein said detecting further comprises plotting a value for a nucleic acid molecule detected in a sample of step II(b).

29. The method of claim 25 wherein said reaction mixture is a PCR reaction mixture.

30. The method of claim 25 wherein said strand displacement activity, measured at optimal polymerizing temperature for said DNA polymerase, is at least as great as the strand displacement activity of Vent Exo⁻™ DNA polymerase measured at 72° C.

31. The method of claim 25 wherein a said oligonucleotide primer is 5′ labeled.

32. The method of claim 31 wherein said label is a fluorescent label.

33. The method of claim 25 wherein said detecting comprises measuring fluorescence.

34. The method of claim 25 wherein said polymerase is at least as processive as Vent Exo⁻™ DNA polymerase.
